(19) United States
(12) Patent Application Publication
(10) Pub. No.: US 2012/0269754 A1
(43) Pub. Date: Oct. 25, 2012

(54) Low Flash Point Lip Composition Containing Solvents of Varying Evaporation Rates

(75) Inventors: Giovana A. Sandstrom, Saddle Brook, NJ (US); John R. Glynn, JR., Ridgewood, NJ (US); Prithwiraj Maitra, Randolph, NJ (US)

(73) Assignee: Avon Products, Inc., New York, NY (US)

(21) Appl. No.: 13/498,484

(22) PCT Filed: Oct. 1, 2010
(86) PCT No.: PCT/US10/51096

§ 371 Date: Mar. 27, 2012

Related U.S. Application Data
(60) Provisional application No. 61/259,248, filed on Nov. 9, 2009.

(51) Int. Cl. A61K 8/91; A61Q 1/04

(52) U.S. Cl. 424/64; 514/772.1

(57) ABSTRACT

Described herein is a cosmetic composition comprising a mixture of volatile solvents with varying evaporation rates, wherein at least two of the volatile solvents are in combination with a film former. A single application of a cosmetic composition as described herein provides transfer resistance, long wear, and comfort and as such, may be used in lipsticks, lip glosses, mascara, hair care products, and other such cosmetics for which transfer resistance, long wear and comfort are desired properties.

### CROSS-REFERENCE TO RELATED APPLICATIONS

This application claims priority to International Application Serial No. PCT/US10/51096 filed Oct. 1, 2010, which claims priority U.S. Provisional Patent Application Ser. No. 61/259,248, filed Nov. 9, 2009, the contents of which are hereby incorporated by reference in their entirety.

### FIELD OF INVENTION

The present invention relates generally to cosmetic compositions which offer transfer resistance, long wear, and comfort with a one step application. More particularly, the present invention relates to cosmetic compositions for a imparting long-wearing, comfortable, transfer-resistant film on the lips comprising a high evaporation rate volatile solvent component, a medium evaporation rate volatile solvent component, a low evaporation rate volatile solvent component, and a silicone acrylate grail copolymer film former that is soluble or dispersible in at least two of the high, medium, and low evaporation rate volatile solvent components.

### BACKGROUND

In the cosmetic industry, transfer resistance, long wear, and comfort are difficult to achieve without trade offs such as tack, particularly in a composition designed for single application. For example, most cosmetic lip compositions comprise film formers dissolved in volatile solvents to achieve such properties. Generally, the volatile solvents used are low evaporation rate volatile solvents such as isododecane, cyclopentasiloxane, methyl trimethicone, etc. In such systems one can only achieve two of the three desired performance attributes of transfer resistance, long wear, and comfort. For example, most cosmetic compositions may exhibit tong wear and transfer resistance, but not comfort, in a single application.

However, there is an ongoing need for cosmetic compositions lipsticks (including lip glosses), mascara, hair care, or other cosmetics) that provide long wear, transfer resistance, and comfort after a single application.

### SUMMARY

Described herein is the unexpected discovery that cosmetic compositions exhibit desired properties such as long wear, transfer resistance, and comfort when the cosmetic compositions comprise an optimal balance of high, medium and low evaporation rate volatile solvent components in combination with at least one silicone acrylate graft copolymer film former that is soluble or dispersible in at least two of the high, medium, and low evaporation rate volatile solvent components. Such cosmetic compositions are particularly useful for imparting a long-wearing, comfortable, transfer-resistant film on the lips, and generally have a flash point of less than about 100° F.

### BRIEF DESCRIPTION OF THE DRAWINGS

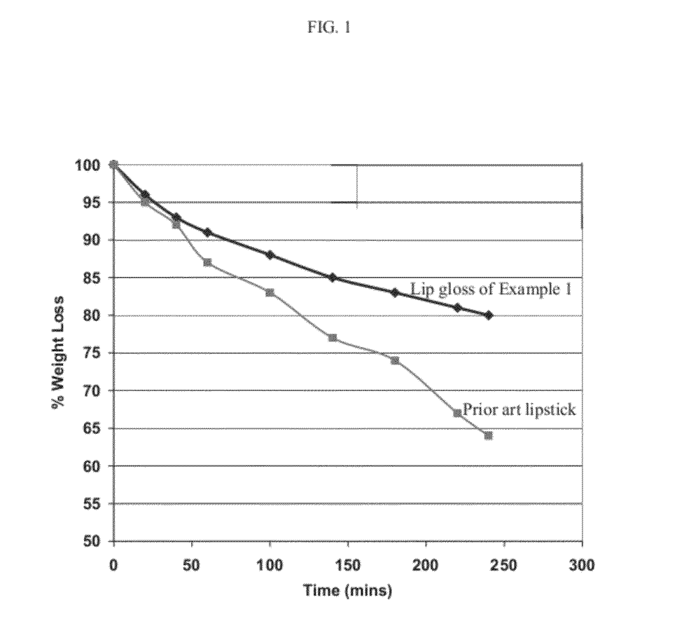

Shown in FIG. 1 is a comparison of a thermogravometric analyzer weight loss curve of a lipstick composition described in Example 1 comprising at least one high evaporation rate volatile solvent component, at least one medium evaporation rate volatile solvent component, and at least one low evaporation rate volatile solvent component (♦) with a thermogravometric analyzer weight loss curve of a prior art lipstick basecoat (▪).

### DETAILED DESCRIPTION

Described herein is a cosmetic composition comprising volatile solvents with varying evaporation rates to provide long wear, transfer resistance, comfort and optionally gloss with a single application, e.g., compositions for lipsticks (including lip glosses), mascara, hair care, or other cosmetics where a transfer resistance, long wear, and comfort are desired after a single application. A cosmetic composition as described herein comprises volatile solvents with varying evaporation profiles such that after product application, the volatile solvents evaporate immediately and/or over a period of few hours.

I. Compositions

A. Solvents

A cosmetic composition as described herein comprises at least one high evaporation rate volatile solvent component, at least one medium evaporation rate volatile solvent component, and at least one low evaporation rate volatile solvent component; wherein each of the volatile solvents are present in amounts to provide long wear, tack, transfer resistance and comfort after application of the cosmetic composition; and wherein at least two of the three solvents further comprise at least one silicone acrylate graft copolymer film former solvated or dispersed therein. Without being bound by theory, it is believed that the high evaporation rate volatile solvent component leads immediately to an instant surface “skin formation” after application, e.g., immediately provides a feel that is dry to touch. It is also believed that the high evaporation rate volatile solvent component improves and may be critical for transfer resistance as an underlying film is forming. However, defects may arise due to the high evaporation rate of such a component. Detects in the film may lead to stress points which affect the long term durability of the film. Accordingly, optimal amounts of medium and/or low evaporation rate volatile solvent components aid and may be critical in controlling shrinkage in the film, which affects a comfortable feel on the lips. The medium evaporation rate volatile solvent component allows long wear film formers to flow (and set), and provide good adhesion to the substrate and transfer resistance properties. The low evaporation rate volatile solvent component allows film formers to make intimate contact with the substrate and also allow the elimination of defects from the film.

A solvent component may be characterized as a high evaporation rate volatile solvent component, a medium evaporation rate volatile solvent component, or a low evaporation rate volatile solvent component based on its properties, e.g., ability to provide a feel that is dry to touch immediately after application (high evaporation rate volatile solvent component), ability to provide transfer resistance (high evaporation rate volatile solvent component and/or medium evaporation rate volatile solvent component), ability to eliminate defects from the film (low evaporation rate volatile solvent component), etc.

Other properties, e.g., evaporation rate, vapor pressure, etc., may be obtained using well-known methods in the art. For example, a thermogravometric analyzer (TGA) may be used to obtain the evaporation rate of a solvent by heating about 10 mg to about 20 mg of the solvent component under constant nitrogen flow from room temperature to 35 at a heating rate of 5° C./minute and holding the solvent component at 35° C., for about 60 to about 120 minutes to determine the percent weight loss.

The compositions described herein comprise at least one high evaporation rate volatile solvent component in combination with at least one medium evaporation rate volatile solvent component and/or at least one low evaporation rate volatile solvent component. In one embodiment, a volatile solvent component may be characterized using a thermogravometric analyzer (TGA), e.g., a Pyris 1 TGA (Perkin Elmer) having a platinum pan with a capacity of 60 μL, according to the manufacturer's protocol. With such a TGA, a high evaporation rate solvent may be characterized as a volatile solvent that exhibits about 20% to about 40% weight loss at 35° C. over 60 minutes. A medium evaporation rate volatile solvent component may be characterized as a solvent that exhibits about 10% to about 20% weight loss at 35° C. over 60 minutes. A low evaporation rate volatile solvent component may be characterized as a solvent that exhibits from about 0.1% to about 10% weight loss at 35° C. over 60 minutes.

Non-limiting examples of high evaporation rate volatile solvent components include hexamethyl disiloxane and/or a silicone fluid having a viscosity of less than 1 cSt at 25° C., including, for example, those silicone fluids having a viscosity of 0.65 cSt. Non-limiting examples of a medium evaporation rate volatile solvent component include mixed dimethicones, e.g., a dimethicone/trisiloxane blend. Non-limiting examples of low evaporation rate volatile solvent components include cyclopentasiloxane, methyl trimethicone, isododecane, and combinations thereof.

Shown in Table 1 are properties (weight loss properties after 60 or 120 minutes at 35° C. and performance properties) that may be used to classify a solvent component as a high evaporation rate volatile solvent component, a medium evaporation rate volatile solvent component or a low evaporation rate volatile solvent component. Also shown in Table 1 are exemplary high evaporation rate volatile solvents, medium evaporation rate volatile solvents, and low evaporation rate volatile solvents, which may be used to form the high evaporation rate volatile solvent component, medium evaporation rate volatile solvent component, and low evaporation rate volatile solvent component as described herein.

TABLE 1

| | Weight loss | Performance | |
|---|---|---|---|
| Rate | (60 minutes)¹ | Properties | Examples |
| High | 20%-40% | Initial Transfer | Hexamethyl disiloxane, |
| | | Resistance, | Dimethicone 0.65 cSt |
| | | “Skinning”, | |
| | | Initial Tack | |
| Medium | 10%-20% | Intermediate | Dimethicone/trisiloxane |
| | | Transfer Resistance, | blend² |
| | | Intermediate | |
| | | Tack, Adhesion | |
| Low | 0.1%-10% | Long Wear Comfort | Cyclopentasiloxane, |
| | | | methyl trimethicone, |
| | | | isododecane, |

¹At 35° C.;
²Dow Corning 2-1184

A cosmetic composition as described herein generally comprises at least one high evaporation rate volatile solvent component, at least one medium evaporation rate volatile solvent component, and at least one low evaporation rate volatile solvent component, each present in amounts to provide an optimal balance of long wear, tack, transfer resistance and comfort, wherein at least two of the three solvents further comprise a film former. In one embodiment, a cosmetic composition described herein comprises at least one high evaporation rate volatile solvent component present in an amount of about 1% to about 75% by total weight of the composition, at least one medium evaporation rate volatile solvent component in an amount of about 1% to about 75% by total weight of the composition, and at least one low evaporation rate volatile solvent component in an amount of about 1% to about 75% by total weight of the composition. In another embodiment, a composition as described herein comprises at least one high evaporation rate volatile solvent component present in an amount of about 2.5 wt % to about 50 wt %, e.g., about 5 wt % to about 25 wt %, by total weight of the compositions. In another embodiment, a composition as described herein comprises at least one medium evaporation rate volatile solvent component present in an amount of about 5 wt % to about 50 wt %, e.g., 10 wt % to about 30 wt %, by total weight of the composition. In another embodiment, a composition as described herein comprises at least one low evaporation rate volatile solvent component present in an amount of about 5 wt % to about 50 wt %, e.g., about 10 wt % to about 30 wt %, by total weight of the composition.

In one embodiment, the total concentration of the three solvent components range from about 5 wt % to about 90 wt % based on the total weight of the composition, e.g., about 25 wt % to about 75 wt % based on the total weight of the composition, e.g., about 40 wt % to about 65 wt % based on the total weight of the composition. The ranges described herein are exemplary, and other ranges of at least one high evaporation rate volatile solvent component, at least one medium evaporation rate volatile solvent component and at least one low evaporation rate volatile solvent component that provide a balance of long wear, tack, transfer resistance, comfort and optionally gloss in a cosmetic composition may be easily determined by a skilled artisan (see, e.g., Example 1). It will be understood that the concentrations of high, medium, and low evaporation rate solvents apply to all solvents in the compositions that meet the volatility requirements, including any amounts of solvents that are introduced along with polymer dispersions or the like.

A cosmetic composition as described herein generally comprises at least one high evaporation rate volatile solvent component, at least one medium evaporation rate volatile solvent component, and at least one low evaporation rate volatile solvent component, each present in amounts to provide an optimal balance of long wear, tack, transfer resistance and comfort, wherein at least two of the three solvents further comprise a film former. In one embodiment, the solvents comprised in the composition are all miscible with each other. In another embodiment, the solvents comprised in the composition are all immiscible with each other. In another embodiment, at least one solvent comprised in the composition is immiscible with another solvent comprised in the composition.

Generally, solvents are liquids to enable easy formulation of a cosmetic composition described herein. When a cosmetic composition described herein is applied to the desired surface, the solvent(s) must be capable of flashing off to leave the other ingredients in the composition affixed to the surface. Generally, the presence of a high evaporation rate volatile solvent component, particularly in an amount of about 5 wt % to about 25 wt % by total weight, leads to a cosmetic composition with a low flash point, which may be critical for the performance properties provided in Table 1. Accordingly, in one embodiment, a cosmetic composition as described herein has a flash point less than 110° F., more typically, less than 100° F., and, in fact, flash points below 90° F. or even below 80° F. have been found to be useful. In one embodiment, a cosmetic composition as described herein has a flash point between about 60° F. and about 100° F. In another embodiment, a cosmetic composition as described herein as a flash point between about 60° F. and about 70° F. In another embodiment, a cosmetic composition as described herein has a flash point between about 70° F. and about 80° F. In another embodiment, a cosmetic composition as described herein has a flash point between about 80° F. and about 90° F. In another embodiment, a cosmetic composition as described herein has a flash point between about 90° F. and about 100° F. Compositions comprising about 15% by weight 0.65 cSt silicone fluid have been found to provide a flash point of about 75-80° F. and composition comprising about 20% by weight 0.65 cSt silicone fluid can provide a flash point of about 60-65° F.

B. Film Forming Polymer

The compositions described herein comprise one or more film-forming polymer for film former) solvated or dispersed in at least two of the three solvents. The term film-forming polymer may be understood to indicate a polymer which is capable, by itself or in the presence of at least one auxiliary film-forming agent, of forming a continuous film and functions as a binder for the particulate material. The film forming polymer may also have adhesive properties, meaning that when incorporated into a composition described herein and applied to a surface, e.g., the lips, lashes, hair, etc., the film forming polymer forms a film or a weld on the surface. Such a film will have adhesive and cohesive strength, as is understood by those skilled in the art. Further, the preferred film forming polymer may preferably form a semi-permanent film on the surface to which it is applied, meaning that the composition containing the polymer is not easily removed from the surface after once it is applied and has adequately dried and set.

Generally, the one or more film-forming polymers may be soluble, dispersible and/or solvated in at least two volatile solvents and when the composition is applied to the desired surface, the volatile solvent(s) at least partially evaporates and causes the film-forming polymer to form a film on the surface which holds the pigment particles in place with the network created by the hardened polymer. The term “soluble” means that the film forming polymer is soluble in its solvent and when combined both components form a homogeneous single phase. The term “dispersible” means that the film forming polymer is readily dispersed in the volatile solvent and forms a stable, heterogeneous composition where the dispersed polymer remains stable in the volatile solvent and is not incompatible therewith.

As described herein, a film-forming polymer only needs to be soluble in at least one volatile solvent comprised in the cosmetic composition, e.g., does not need to be capable of dissolution in each solvent comprised in the cosmetic composition. Cosmetic compositions as described herein may contain hydrocarbon and silicone solvents with a wide range of evaporation rates. The range of evaporation rates based upon n-butyl acetate may be from 0.01 to 1.0.

Water-soluble, water-dispersible, or water-insoluble film formers, including film-forming polymers, may be employed. Polymeric film formers include, without limitation, acrylic polymers or co-polymers, acrylates, polyolefins, polyvinyls, polyacrylates, polyurethanes, silicones, polyamides, polyethers, polyesters, fluoropolymers, polyethers, polyacetates, polycarbonates, polyamides, polyimides, rubbers, epoxies, formaldehyde resins, organosiloxanes, dimethicones, methicones, cellulosics, polysaccharides, polyquaterniums, and the like. Suitable film formers include those listed in the Cosmetic Ingredient Dictionary (INCI) and Handbook, 12^{th} Edition (2008), the disclosure of which is hereby incorporated by reference.

In a preferred embodiment, film-forming polymers as described herein are the reaction products of ethylenically unsaturated monomers having carboxylic acid or esters functional groups. Such monomers may have the following structure:

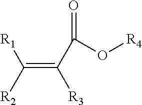

where R₁, R₂, and R₃ are independently selected from hydrogen or a C₁-C₁₀ hydrocarbon radical, including, for example, methyl, ethyl, propyl, iso-propyl, n-butyl, iso-butyl, tert-butyl, pentyl (amyl), hexyl, cyclohexyl, heptyl, octyl, phenyl, benzyl, and the like. In some monomers R₁, R₂ and R₃ will be hydrogen. Such monomers are known generally as acrylic acid or acrylate monomers. In other monomers R₁ and R₂ will be hydrogen and R₃ will be methyl. Such monomers will be recognized as methacrylic acid or methacrylate monomers. The term (meth)acrylic or (meth)acrylate refers to the situation where R₃ can be either hydrogen or methyl. The term (alkyl)acrylic or (alkyl)acrylate refers to the situation where R₃ can be either hydrogen or alkyl. That is, the parenthetical, in each case, indicates that the alkyl substitution is optional.

R₄ is independently selected from hydrogen or a C₁-C₁₀ hydrocarbon radical, including, for example, methyl, ethyl, propyl, iso-propyl, n-butyl, iso-butyl, tert-butyl, pentyl (amyl), hexyl, cyclohexyl, heptyl, octyl, phenyl, benzyl, and the like. Any of the foregoing groups can be straight chained or branched and may optionally include one or more points of unsaturation. These hydrocarbon radicals may also optionally be substituted with one or more heteroatoms, including halogen (F, Cl, Br, I), oxygen, nitrogen, sulfur, and the like. Perhalo-derivatives are also contemplated, including perfluoro-derivatives such as trifluoromethyl.

The film forming polymers of the invention may be graft copolymers and may include one or more monomers having organosilicone polymers grafted onto a hydrocarbon group. R₄ may be, for example, a moiety of the form:

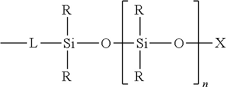

where L is a linker comprised of a di-valent C₁-C₂₀ hydrocarbon radical, such as methylene (—CH₂—), ethylene (—CH₂CH₂—), propylene (—CH₂CH₂CH₂—), butylene (—CH₂CH₂CH₂CH₂—), or the like; R is independently selected from hydrogen or a C₁-C₁₀ hydrocarbon radical, including, for example, methyl, ethyl, propyl, iso-propyl, n-butyl, iso-butyl, tert-butyl, pentyl (amyl), hexyl, cyclohexyl, heptyl, octyl, phenyl, benzyl, and the like; and X is a capping group which may be, for example, any of the foregoing hydrocarbon radicals, or may be a group —Si(R)₃ where R is independently selected at each occurrence and is preferably methyl at each occurrence; and n is an integer from 1 to 200; typically from 5 to 100, or from 10 to 50.

In some embodiments, R is selected independently at each occurrence from hydrogen, hydroxyl, and optionally substituted hydrocarbon groups containing from 1 to 10 carbon atoms, and in particular from optionally substituted alkyl, alkenyl, alkynyl, aryl, alkyl-aryl, or aryl-alkyl groups; preferably R is selected from optionally substituted branched, straight chain, or cyclic C₁₋₆ alkyl or alkenyl groups, including without limitation, methyl, ethyl, propyl, isopropyl, butyl, isobutyl, t-butyl, amyl, hexyl, cyclohexyl, vinyl, allyl, and the like or C₁₋₈ aryl, alkyl-aryl, or aryl-alkyl groups, including without limitation, phenyl, benzyl, tolyl, xylyl and the like;

wherein each of the foregoing R groups may include optional substitution by one or more heteroatoms, including oxygen, nitrogen, phosphorous, and halogen, particularly fluorine, as exemplified by fluoroalkyl (including perfluoroalkyl) groups, such as mono-, di-, and tri-fluoromethyl, perfluorophenyl, and the like, amino-substituted C₁₋₆ alkyl groups, including those having the form —(CH₂)₁₋₆—NR^{N}₂ and —(CH₂)₁₋₆—NR^{N}—(CH₂)₁₋₆—NR^{N}₂ where R^{N} is typically hydrogen, but may be methyl, ethyl, propyl, and the like; polyether groups including without limitation, polyethyleneoxide groups of the form —(CH₂CH₂O)ₙ—, polypropylene oxide groups of the form —(CH(CH₃)CH₂O)ₙ— and combinations thereof; and amine oxide, phosphate, hydroxyl, ester, and/or carboxylate functionalities, and the like;

In a preferred embodiment R₄ comprises a dimethicone polymer of the form:

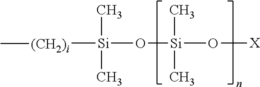

where i is an integer from 1 to 20, typically from 1 to 10, and preferably is 2, 3, 4, 5, or 6; and X is a chain terminating group, which may be, for example, hydrogen, alkyl, or —Si(R)₃ where R is independently selected at each occurrence from the R groups listed above, but is preferably methyl at each occurrence; and n is an integer from 1 to 200, typically from 5 to 100, and preferably from 10 to 50, and more preferably from 15 to 35.

Any of the foregoing hydrocarbon groups can be straight-chained or branched and may optionally include one or more points of unsaturation. The hydrocarbon radicals may also optionally be substituted with one or more heteroatoms, including halogen (F, Cl, Br, I), oxygen, nitrogen, sulfur, and the like. Perhalo-derivatives are also contemplated, including perfluoro-derivatives such as trifluoromethyl. The silicone polymer portion of R₄ may comprise, for example, methicone, dimethicone, amodimethicone, C₁-C₆ organosilicone, C₁-C₆ diorganosilicone, hydroxyalkylsilicone (e.g., hydroxypropyldimethicone), alkyl-arylsilicone, or like polymeric regions.

The polymers of the invention may be the reaction products of one, two, three, four, or more different ethylenically unsaturated monomers. In one embodiment the polymer is the reaction product of at least one acrylate monomer, at least one methacrylate monomer, and at least one (meth)acrylate monomer having a grafted silicone polymer in R₄.

The ethylenically unsaturated monomers having carboxylic acid or esters functional groups will typically comprise greater than about 50%, more typically greater than about 75%, preferably greater than about 85%, more preferably greater than about 90%, and more preferred still greater than about 95% of the monomers from which the film forming polymer is formed. In other embodiments, the film forming polymer will consist of or consist essentially of the reaction product of ethylenically unsaturated monomers having carboxylic acid or esters functional groups. By “consist essentially of” the reaction product of ethylenically unsaturated monomers having carboxylic acid or esters functional groups is meant that the polymer will not contain, or will contain such low levels of other monomers (excluding end or capping groups), such as ethylenically unsaturated monomers which do not comprise carboxylic acid or esters functional groups, that the properties of the polymer (e.g., transfer resistance) are not measurable impacted.

The compositions comprise one or more film formers derived from acrylic acid, and may suitably include, for example, two or three chemically distinct film formers derived from acrylic acid.

Specific mention may be made of silicone acrylate copolymers, including without limitation, those having the INCI names Butyl Acrylate/Hydroxypropyl Dimethicone Acrylate Copolymer (CTFA Monograph ID 12998), Acrylates/Dimethicone Copolymer (CTFA Monograph ID 10082), Acrylates/Ethylhexyl Acrylate/Dimethicone Methacrylate Copolymer (CTFA Monograph ID 16592), combinations thereof, etc. In a preferred embodiment, the acrylate film firmer selected from the group consisting of Butyl Acrylate/Hydroxypropyl Dimethicone Acrylate Copolymer (CTFA. Monograph ID 12998), Acrylates/Dimethicone Copolymer (CTFA Monograph ID 10082), and combinations thereof. Silicone acrylate polymers may suitably be solvated in an appropriated solvent, such as methyl trimethicone, isododecane (IDD), dimethylsiloxane, cyclodimethicone pentamer/hexamer, or the like. It is contemplated that the use of silicone acrylate copolymers may impart both water and oil transfer resistance. Other suitable film formers may not comprise silicone polymers. Non-limiting examples of specific film formers derived from acrylic acid that are not silicone copolymers include, without limitation, Acrylates Copolymer (CTFA Monograph ID 52).

Generally, the film former is present in an amount from about 0.1 wt % to about 85 wt % by total weight of the composition. Typically, the film former is present from about 1% to about 75% by weight, more typically between about 5% and about 50%, and preferably, between about 10% and about 30% by weight, based on the total weight of the composition. Combinations of any of the foregoing film formers are also contemplated to be suitable, including combinations of (i) a film former having a high glass transition temperature (high Tg film former) and (ii) a film former having a low glass transition temperature (low Tg film former). As used herein, a high Tg film former has a Tg of ≧37° C., whereas a low Tg film former has a Tg <37° C.

Shown in Table 2 are typical, preferred, and most preferred ranges (wt % by total weight of the composition) of high Tg and low Tg film formers and high, medium, and low evaporation rate volatile solvent components that may be used in a cosmetic composition as described herein.

TABLE 2

| | Most preferred | Preferred | Typical |
|---|---|---|---|
| High Tg film former | 10-16 | 10-20 | 10-25 |
| Low Tg film former | 8-12 | 8-15 | 8-25 |
| High evaporation rate volatile | 5-25 | 2.5-50 | 1-75 |
| solvent component | | | |
| Medium evaporation rate volatile | 10-30 | 5-50 | 1-75 |
| solvent component | | | |
| Low evaporation rate volatile | 10-35 | 5-50 | 1-75 |
| solvent component | | | |

In one embodiment, a composition as described herein comprises a film former solvated and/or dispersed in at least two volatile solvents, wherein the two volatile solvents have different evaporation rates. In a preferred embodiment, a composition as described herein (e.g., a tip composition) comprises a silicone acrylate copolymer film former, wherein the silicone acrylate copolymer film former is solvated and/or dispersed in at least two volatile solvents having different evaporation rates; wherein the two solvents having different evaporation rates are present in amounts effective to provide an optimal balance of long wear, tack, transfer resistance and comfort. In one embodiment, the silicone acrylate copolymer film former is selected from the group consisting of Butyl Acrylate/Hydroxypropyldimethicone Acrylate Copolymer, Acrylate/Dimethicone Copolymer, Acrylates/Ethylhexyl Acrylate/Dimethicone Methacrylate, and a combination thereof. In another embodiment, the at least two volatile solvents having different evaporation rates are a high evaporation rate volatile solvent component and a medium evaporation rate volatile solvent component. In another embodiment, the high evaporation rate volatile solvent component comprises a silicone fluid dimethicone) having a viscosity of 0.65 cSt and the medium evaporation rate volatile solvent component is a dimethicone/trisiloxane blend (e.g., 2-1184 from Dow Corning), wherein the dimethicone 0.65 cSt and solvated or dispersed silicone acrylate film former is present in an amount of about 40 wt % to about 50 wt % by total weight of the composition, and wherein the dimethicone/trisiloxane blend and solvated or dispersed silicone acrylate film former is present in an amount of about 20 wt % to about 30 wt % by total weight of the composition. In another embodiment, the lip composition further comprises a low evaporation rate volatile solvent component, e.g., cyclopentasiloxane, methyl trimethicone, isododecane, and a combination thereof.

Furthermore, a cosmetic composition as described herein may have a viscosity of less than about 25,000 centipoise (cps), e.g., less than about 15,000 cps, e.g., less than about 10,000 cps at 25° C. This viscosity range may generally translate into a film thickness that improves long wear, reduces tack and provides comfort on the lips.

C. Other Ingredients

A compositions as described herein may further comprise other ingredients as is customary in a given product. It is understood that the weight percents (by total weight of the composition) of the volatile solvent(s), film-forming polymer(s), and other ingredients in a composition described herein shall not exceed 100%.

1. Colorants and Fillers

The cosmetic compositions may optionally further comprises various colorants (e.g., pigments, pearlescents, dyes, lakes, etc.) and fillers, as is customary in a given product. These include, without limitation, metal oxide pigment such as iron oxides and titanium dioxide, silica, alumina, nylon powder. Teflon powder, PMMA, silicone elastomers, and the like. For other pigments, lakes and dyes used in cosmetic industry as colorants, refer to the Cosmetic Ingredient Dictionary (INCI) and Handbook, 12^{th} Edition (2008), published by the Cosmetic, Toiletry, and Fragrance Association (CTFA), the disclosure of which is hereby incorporated by reference. Such pigments, filters and the like will typically comprise from about 0.1 wt % to about 20 wt % by weight of the composition, more typically from about 0.8 wt % to about 10 wt % by weight of the composition.

In one embodiment, a cosmetic composition as described herein comprises a particulate colorant, which comprises (i) a particulate modifying agent; and (ii) a first colorant bonded to the surface of the particulate modifying agent. As used herein, the term “colorant” generally refers to a color extender, dye, pigment, lake, toner, other agent, or a combination thereof, used to impart a color to a material, and includes inorganic, organic, water-soluble and water-insoluble substances. As used herein, the term “modifying agent” includes a substrate responsible for imparting additional optical or visual properties to the material.

The particulate colorant may be formed according to the procedures described in Sensient Colors Inc.'s U.S. Patent Pub, 2007/0020208, the disclosure of which is hereby incorporated by reference herein. For example, the particulate colorant may be prepared by blending, either in dry form or as slurries or solutions, the first colorant with the particulate modifying agent. The first colorant may be bonded to the surface of the particulate modifying agent by, for example, adding a surface treatment to the dry blend. By “bonded” is meant chemical bonding through strong interactions, for example, ionic or covalent bonds, or by physical bonding through weak interactions, for example, by dipole-dipole interactions such as hydrogen bonds, charge-transfer complexes, hydrophobic interactions, van der Waals forces, or combinations thereof.

The modifying agent may be, without limitation, a metal oxide, such as aluminum oxide (alumina), zinc oxide, silicon dioxide (silica), magnesia, or a combination thereof; talc; mica; kaolin; bismuth oxychloride; stainless steel; graphite; or platy metals such as bronze, copper and aluminum or a combination thereof. A preferred modifying agent comprises a metal oxide; in particular alumina. The modifying agent may be in the shape of a platelet, for example, a platelet of alumina. As used herein, the term “platelet” generally refers to a substantially planar and flaky material that is generally not spherical and is greater in width and length than in thickness. For example, suitable platelets may have an average diameter of between 1 and 20 microns, and an average thickness less than 0.5 microns. In some embodiments, the edge of the platelet is substantially free of colorant, by which is meant that at least 90% of the total surface area of the edge of the modifying agent has no colorant adhered or bonded to it. The top and bottom faces of the platelet will typically have the first colorant adhered to about 5% to about 90% of their surface area. In some embodiments, the first colorant may cover or coat more than about 1%, 3%, 5%, 10%, 15%, 20%, 30%, 40% or 50% of the total surface area of the modifying agent and less than about 99%, 95%, 90%, 85%, 80%, 75%, 65%, 50%, 40%, 30%, 25%, 20%, 15% or 10%, of the total surface area of the modifying agent. According to Sensient Colors Inc.'s U.S. Patent Pub. 2007/0020208, these particulate colorants may exhibit increased burnishing when compared with platelet alumina completely coated with colorant.

The first colorant may comprise, for example, an inorganic pigment. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (α-Fe₂O₃, γ-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides and composite oxides and composite hydroxides such as iron titanate, cobalt titanate and cobalt aluminate. Non-metal oxides such as alumina and silica, ultramarine blue sodium aluminum silicate containing sulfur), Prussian blue, manganese violet, bismuth oxychloride, talc, mica, sericite, magnesium carbonate, calcium carbonate, magnesium silicate, aluminum magnesium silicate, silica, titanated mica, iron oxide titanated mica, bismuth oxychloride, and the like, are also contemplated to be suitable inorganic pigments.

The first colorant may comprise, for example, an organic pigment. Organic pigments can include, but are not limited to, at least one of carbon black, carmine, phthalocyanine blue and green pigment, diarylide yellow and orange pigments, and azo-type red and yellow pigments such as toluidine red, litho red, naphthol red and brown pigments, and combinations thereof.

The first colorant component may comprise, for example, one or more dyes, toners or lakes. Lakes generally refer to a colorant prepared from a water-soluble organic dye (e.g., D&C or FD&C) which has been precipitated onto an insoluble reactive or adsorptive substratum or diluent. The term “D&C” means drug and cosmetic colorants that are approved for use in drugs and cosmetics by the FDA. The term “FD&C” means food, drug, and cosmetic colorants which are approved for use in foods, drugs, and cosmetics by the FDA. Certified D&C and FD&C colorants are listed in 21 C.F.R. §74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B. Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 0.4, Blue 6, Green 5, Green 6, Green 8, Orange 4, Orange 5, Orange 10, Orange 11, and so on.

Substrates suitable for forming lakes include, without limitation, mica, bismuth oxychloride, sericite, alumina, aluminum, copper, bronze, silver, calcium, zirconium, barium, and strontium, titanated mica, fumed silica, spherical silica, polymethylmethacrylate (PMMA), micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, nylon, silica silylate, silk powder, sericite, soy flour, tin oxide, titanium hydroxide, trimagnesium phosphate, walnut shell powder, and mixtures thereof.

Suitable lakes include, without limitation, those of red dyes from the monoazo, disazo, fluoran, xanthene, or indigoid families, such as Red 4, 6, 7, 17, 21, 22, 27, 28, 30, 31, 33, 34, 36, and Red 40; lakes of yellow pyrazole, monoazo, fluoran, xanthene, quinoline, dyes or salt thereof, such as Yellow 5, 6, 7, 8, 10, and 11; lakes of violet dyes including those from the anthroquinone family, such as Violet 2, as well as lakes of orange dyes, including Orange 4, 5, 10, 11, and the like. Suitable Lakes of D&C and FD&C dyes are defined in 21 C.F.R. §82.51.

The surface treatment may be any such treatment that modifies the surface of the modifying agent. For example, the surface treatment may make the particles more hydrophobic or more dispersible in a vehicle or may increase the adhesion of the first colorant to the modifying agent. The surface of the particles may, for example, be covalently or ionically bound to an organic molecule or silicon-based molecule or may be adsorbed thereto, or the particle may be physically coated with a layer of material. The surface treatment compound may be attached to the particle through any suitable coupling agent, linker group, or functional group (e.g., silane, ester, ether, etc). The compound may comprise a hydrophobic portion which may be selected from, for example, alkyl, aryl, allyl, vinyl, alkyl-aryl, organosilicone, di-organosilicone, dimethicones, methicones, polyurethanes, silicone-polyurethanes, and fluoro- or perfluoro-derivatives thereof. Other hydrophobic modifiers include lauroyl lysine, Isopropyl Titanium Triisostearate (ITT), ITT and Dimethicone (ITT/Dimethicone) cross-polymers, ITT and Amino Acid, ITT/Triethoxycaprylylsilane Crosspolymer, waxes (e.g., carnauba), fatty acids (e.g., stearates), HDI/Trimethylol Hexylactone Crosspolymer, PEG-8 Methyl Ether Triethoxysilane, aloe, jojoba ester, lecithin, Perfluoroalcohol Phosphate, and Magnesium Myristate (MM), to name a few.

The surface treatment may comprise, in some embodiment, a material selected from aluminum laurate, aluminum stearate, an amino acid, chitin, collagen, fluorochemical, metal soap, natural wax, polyacrylate, polyethylene, silicone, slime, titanatate ester, urethane, dimethicone, perfluoropolymethylisopropyl ether, styrene acrylates copolymer, magnesium myristate, lauroyl lysine and a combination thereof. In other embodiments, the surface treatment comprises a material selected from methicone, triethoxycaprylylsilane, trimethoxycaprylyisilane, dimethicone copolyol and a combination thereof.

In one embodiment, the particulate colorant has been surface treated with an alkylsilane, such as a C₁₋₂₀ alkylsilane, or more typically a C₁₋₁₂ alkylsilane, including an exemplary embodiment wherein the particle is surface-treated with a C₈ alkylsilane (e.g., caprylylsilane). The colorants may be prepared, for example, by treating a particulate with a trialkoxyalkylsilane, such as Triethoxycaprylylsilane (INCI).

in another embodiment, the particulate has been surface treated with a fluoroalkylsilane, and in particular a perfluoroalkylsilane, such as a C₁₋₂₀ perfluoroalkylsilane, or more typically a C₁₋₁₂ perfluoroalkylsilane, including an exemplary embodiment wherein the particulate colorant is surface-treated with a C₈ perfluoroalkylsilane. These may be prepared by treating a particulate colorant with a trialkoxyfluoroalkylsilane, such as Perfluorooctyl Triethoxysilane (INCI). An example of such a compound is tridecafluorooctyltriethoxy silane, available from Sivento, Piscataway, N.J., under the trade name DYNASILANE™ F 8261.

In some embodiments, the alkyl silane surface-treated colorant consists essentially of or comprises an alumina substrate (e.g., platelet shaped) and a pigment, dye, or lake bonded to the alumina substrate by an alkyl silane surface treatment. Typically, the alkyl silane will be octylsilane and may be formed by treatment with Triethoxy Caprylylsilane. Nonlimiting examples of such colorants include, but are not limited to, Alumina/Titanium Dioxide/Triethoxycaprylylsilane 1% (COVALUMINE™ Atlas White AS), Alumina/D&C Red Aluminum Lake CTD/Triethoxycaprylylsilane 1% (COVALUMINE™ Red Rose AS), Alumina/D&C Red Aluminum Lake CTD/Triethoxycaprylylsilane 1% (COVALUMINE™ Sonoma Red AS), Alumina/Black Iron Oxide CTD/Triethoxycaprylylsilane 1% (COVALUMINE™ Sonoma Black AS), Alumina/D&C Red #6 Aluminum Lake CTD/Triethoxycaprylylsilane 1% (COVALUMINE™ Fire Red AS), Alumina/Yellow iron Oxide CTD/Triethoxycaprylylsilane 1% (COVALUMINE™ Sonoma Yellow AS), Alumina/D&C Blue #1 Aluminum Lake CTD/Triethoxycaprylylsilane 1% (COVALUMINE™ Astral Blue AS), Alumina/Carmine CTD/Triethoxycaprylylsilane 1% (COVALUMINE™ Campari AS), Alumina/Yellow #5 CTD/Triethoxycaprylylsilane 1% (COVALUMINE™ Sunburst AS), Alumina/Triethoxycaprylylsilane 1%, and combinations thereof, each of which is available from SENSIENT™ Cosmetic Technologies LCW.

In one embodiment, a cosmetic composition as described herein comprises a total of about 0.1% to about 75% by weight of the particulate colorant component, based on the total weight of the composition. Typically, the particulate colorant component will comprise from about 0.5% to about 50% by weight, more typically from about 1% to about 40% by weight, and preferably from about 2% to about 30% by weight of the total composition. In other embodiments the particulate colorant component will comprise from about 3% to about 25% by weight, more typically from about 4% to about 15% by weight, and preferably from about 5% to about 10% by weight of the total composition.

Suitable fillers include without limitation silica, treated silica, talc, zinc stearate, mica, kaolin, Nylon powders such as Orgasol™, polyethylene powder, Teflon™, starch, boron nitride, copolymer microspheres such as Expancel™ (Nobel Industries), Polytrap™ (Dow Corning) and silicone resin microbeads (Tospearl™ from Toshiba), and the like. Other suitable particulates include the particulate silicon wax sold under the trade name Tegotop™ 105 (Degussa/Goldschmidt Chemical Corporation) and the particulate vinyl polymer sold under the name Mincor™ 300 (BASF).

Other pigment/powder fillers include, but are not limited to, inorganic powders such as gums, chalk, Fuller's earth, kaolin, sericite, muscovite, phlogopite, synthetic mica, lepidolite, biotite, lithia mica, vermiculite, aluminum silicate, starch, smectite clays, alkyl and/or trialkyl aryl ammonium smectites, chemically modified magnesium aluminum silicate, organically modified montmorillonite clay, hydrated aluminum silicate, fumed aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicone dioxide, and boron nitride; organic powder such as polyimide resin powder (nylon powder), cyclodextrin, methyl polymethacrylate powder, copolymer powder of styrene and acrylic acid, benzoguanamine resin powder, polyethylene tetrafluoride) powder, and carboxyvinyl polymer, cellulose powder such as hydroxyethyl cellulose and sodium carboxymethyl cellulose, ethylene glycol monostearate; inorganic white pigments such as magnesium oxide; and stabilizers/rheology modifiers, for example, Bentone Gel and Rheopearl TT2. Other useful powders are disclosed in U.S. Pat. No. 5,688,831, the disclosure of which is hereby incorporated by reference, or any of the clays disclosed in EP 1 640 419, the disclosure of which is hereby incorporated by reference.

Filler/pigments also include hydrophobically modified fumed silica (SiO₂) particles including, but not limited to AEROSIL™ R 202, AEROSIL™ R 805, AEROSIL™ R 812, AEROSIL™ R 812 S, AEROSIL™ R 972, AEROSIL™ R 974, AEROSIL™ R 8200, AEROXIDE™ LE-1, AEROXIDE™ LE-2, and AEROXIDE™ LE-3 from Degussa Corporation of Parsippany, N.J. While silica (SiO₂) and hydrophobically-modified silicas are contemplated to be useful in some embodiments, in other embodiments the compositions will be substantially free of silica or hydrophobically-modified silica, by which is meant that these components comprise less than about 2%, preferably less than about 1%, and more preferably less than about 0.5% by weight of the one or more particulate materials. A suitable hydrophobically modified alumina particulate is ALU C 805 from Degussa.

2. Shine Agents

The cosmetic compositions of the invention may optionally include one or more agents that provide or enhance shine. Shine enhancing agents will typically have a refractive index greater than about 1.4, preferably greater than about 1.5 when measured as a film at 25° C. Suitable shine enhancing agents include without limitation, polyols, fatty esters, silicone phenylpropyldimethylsiloxysilicate, polybutene, polyisobutene, hydrogenated polyisobutene, hydrogenated polycyclopentadiene, propyl phenyl silsesquioxane resins; lauryl methicone copolyol, perfluorononyl dimethicone, dimethicone/trisiloxane, methyl trimethicone, and combinations thereof. In one embodiment, the composition will comprise a shine-enhancing agent in an amount from about 0.1% to about 10% by weight, more preferably from about 1% to about 5% by weight, based on the total weight of the composition.

3. Waxes

The cosmetic compositions of present invention may optionally include one or more waxes. The one or more waxes can be natural (e.g., vegetable, animal, or mineral) waxes or synthetic waxes (e.g., polyolefine, Fisher Tropsch, etc.). A preferred wax is microcrystalline waxes, which will preferably be composed of C₈ to C₅₀ hydrocarbons and will have a melting point preferably greater than about 60° C. Other waxes that may be mentioned include, without glyceryl tribehenate, candelilla, carnuaba, ozokerite, paraffin, polyethylene, beeswax, ceresin, hydrogenated castor oil, japan wax, and mixtures thereof. In one embodiment, the amount of wax is less than about 2 wt % of the total weight of the composition. In another embodiment, the amount of wax ranges from about 0.1% to less than about 2% by weight based on the total weight of the composition. However, more wax, e.g., up to 7.5 wt % based on the total weight of the composition, can be used if clarity is not a concern.

4. Cosmetically Acceptable Vehicles

The compositions will typically comprise a cosmetically acceptable vehicle. By “cosmetically acceptable” is meant that the vehicle is safe for contact with human skin. It is contemplated that any cosmetically acceptable vehicle known in the art will be useful. The vehicle may comprise water, hydrophobic, and/or hydrophilic solvents.

Suitable hydrophilic solvents include but are not limited to, water, isopropyl alcohol, ethyl alcohol, glycerin, butylene glycol, propylene glycol, pentylene glycol, caprylyl glycol, polyglycerol diisostearate, dimethylsiloxane/glycol copolymer, isopropyl myristate, triisostearyl citrate, or any combinations thereof. Suitable hydrophobic vehicles include volatile or non-volatile hydrocarbon oils, silicones, fatty ester oils, and the like.

The compositions of the invention may, in some embodiments, be provided as anhydrous formulations. By “anhydrous” is mean that the weight percentage of water in the composition is less than about 1% by weight. Preferably, the anhydrous compositions are substantially free of water by which is meant that water is not deliberately added to the compositions and the level of water is no more than would be expected based on the absorption of water from the air.

The vehicle may comprise from about 5% to about 99% by weight of the composition, typically from about 30% and about 90% by weight, and more typically from about 50% and about 70% by weight of the composition.

5. Emulsions

The compositions according to the invention may be formulated as water-in-oil (W/O) emulsions, oil-in-water (O/W) emulsions, water-in-silicone, silicone-in-water emulsions, and the like. These emulsions comprise a continuous phase and a discontinuous phase. The continuous phase may be aqueous, oil-based, or silicone-based and the discontinuous phase may likewise be aqueous, oil-based, or silicone-based, depending on the nature of the continuous phase. Combined oil and silicone phases are also possible.

The oil phase may comprise any of the hydrophobic oils discussed herein, including, without limitation, vegetable oils; fatty acid esters; fatty alcohols; isoparaffins such as isododecane; silicone oils such as dimethicones, cyclic silicones, and polysiloxanes; hydrocarbon oils such as mineral oil, petrolatum, isoeicosane and polyisobutene; natural or synthetic waxes; and the like.

The emulsions will typically comprise an amount of emulsifier sufficient to stabilize the emulsion. The amount of emulsifier will typically be from about 0.001 wt % to about 20 wt %, but preferably will range from about 0.01 to about 10 wt %, and most preferably about 0.1 wt % to about 5 wt %, based upon the total weight of the composition.

6. Emollients

The cosmetic compositions may optionally comprise one or more emollients in an amount from about 0.1% up to about 20% by weight, based on the total weight of the composition. More typically, emollients will be present in an amount from about 2 wt % to about 15 wt %, preferably, about 5 wt %. Emollients useful in the present invention include any known to the art, including, but not limited to, oils and esters, such as lanolin and petrolatum. Other emollients include jojoba oil, lanolin oil, coconut oil, palm kernel glycerides, grape seed oil, evening primrose oil, sesame oil, castor oil, meadowfoam seed oil, emu oil, dimethicone copolyol meadowfoamate, wheat germ oil, macadamia nut oil, avocado oil, and mixtures thereof.

7. Thickeners

The composition may comprise a thickener, such as vegetable gums, carboxymethyl cellulose, silica, acrylic acid polymers, clays, such as hectorites, bentonites, hydrated magnesium and aluminium silicates, or calcium silicates, or the like. When present, thickeners will comprise from about 0.1% to about 15% by weight of the composition, more typically from about 1% to about 5% by weight of the composition.

8. Other Ingredients

The composition may comprise one or more preservatives such as methyl, ethyl, or propyl paraben, and so on, in amounts ranging from about 0.0001 wt %-5 wt % by weight of the total composition. The compositions may have other ingredients such as one or more anesthetics, anti-allergenics, antifungals, anti-inflammatories, antimicrobials, antiseptics, chelating agents, emollients, emulsifiers, fragrances, humectants, lubricants, masking agents, medicaments, moisturizers, pH adjusters, preservatives, protectants, soothing agents, stabilizers, sunscreens, surfactants, thickeners, viscosifiers, vitamins, or any combinations thereof.

In addition, a cosmetic composition according to the present invention may comprise other ingredients and additives known in the art, depending on the purpose for which the cosmetic is intended. For example, a composition described herein may optionally include one or more functional agents, fillers and fragrances.

The compositions according to the invention may be useful in a variety of cosmetic and personal care products, including without limitation, lipsticks, and lipcolors, lip gloss, mascaras, transfer-resistant foundations, eyeliner, eyeshadow, water-proof sunscreens and insect repellents, skin care products, hair care products, antiperspirants and deodorants, and other cosmetic products where transfer resistant films are desired.

In another embodiment, the invention is formulated in a conventional lipstick or lipcolor product and may include, without limitation, any of the components disclosed in U.S. Pat. No. 6,509,009, U.S. Pat. No. 6,428,797, U.S. Pat. No. 6,261,576, U.S. Pat. No. 5,747,017, U.S. Pat. No. 5,318,775, and U.S. Pat. No. 4,935,228, the disclosures of which are hereby incorporated by reference.

II. Application

A composition according to the invention is expected to achieve transfer resistance, comfort, and optionally gloss, and remain on the skin or hair for a long-wear period such as from about 1 to about 24 hours. The composition is also expected to maintain transfer resistance and/or long-wear properties for a long-wear period such as from about 1 hour to about 24 hours. Typically, a long-wear period may be from about 8 to about 24 hours. Preferably, the long-wear period may be from about 8 to about 12 hours. An alternative preferred long-wear period may be from about 12 to about 24 hours.

A variety of evaluation methods of transfer resistance and long wearing properties are known in the cosmetic arts, such as in dry blot, oil blot, and rub tests. For example, U.S. Pat. No. 6,071,503 discloses various methods of evaluating cosmetic properties, the disclosure of which is hereby incorporated by reference.

A cosmetic composition as described herein provides transfer resistance, long wear, comfort, and optionally gloss, after a single application, e.g., erosion of the cosmetic off the lips, (e.g., from drinking, eating, smoking, talking), eyelashes (e.g., from waters, tears, sweat, etc.) or skin and the like is significantly reduced as is the need for multiple application of cosmetic composition.

Accordingly, also provided is a method for imparting long-wear and transfer-resistant color (and/or gloss) to a surface of interest comprising applying to the surface a cosmetic composition comprising (i) about 1 to about 75% by weight of a high evaporation rate volatile solvent component comprising hexamethyl disiloxane, a silicone fluid having a viscosity of less than 1 cSt at 25° C., or a combination thereof; (ii) about 1 to about 75% by weight of a medium evaporation rate volatile solvent component; (iii) about 1 to about 75% by weight of a low evaporation rate volatile solvent component; and (iv) about 0.1 wt % to about 85 wt % by weight of a silicone acrylate graft copolymer film former that is soluble or dispersible in at least two of the high, medium, and low evaporation rate volatile solvent components; wherein the cosmetic composition has a flash point of less than about 100° F.

A surface of interest as described herein may be selected from the group consisting of the lip, e.g., the upper and/or lower lip, eyelash(es), hair(s), nail(s) and skin. In one embodiment, applying a cosmetic composition as described herein comprises coating the entire surface of interest with the cosmetic composition using a single stroke. Coating as used herein refers to the administration, affixation, placement, spreading, laying, etc., of the cosmetic composition on the surface of interest. After a single stroke application, the cosmetic composition dries to an even film on the surface of interest, and resists transfer and wear from abrasion. Accordingly, a second layer of the cosmetic composition need not be applied, and is preferably not applied, within a short duration of time after application of the first layer, e.g., is not applied at least until after the composition dries to an even film.

III. Storage and Packaging

A composition as described herein may be formulated as a lipstick, a lip gloss, mascara, eyeshade, eyeliner, foundation, a hair care product, or other flowable cosmetic where a transfer resistance, long wear, gloss and comfort are desired after a single application, and may be stored and/or packaged in any suitable device for the cosmetic. Such storage devices are well-known in the art, see, e.g., U.S. Pat. No. 6,987,537,406. As a nonlimiting example, a lip gloss composition as described herein may be stored and/or packaged in a container (e.g., a cylindrical container) wherein the lip gloss composition is stored in the container and is applied to the lips with an applicator, e.g., a doe foot applicator, which may be affixed to a cap/rod/applicator assembly. In another nonlimiting example, a mascara composition as described herein may be stored and/or packaged in a tubular container that is open at one end. An applicator comprising a rod having bristles at one end and a cap portion at its other end may be removably disposed in the container and in contact with the mascara composition in the container. Typically the rod and bristles are housed or submerged directly in the mascara composition. The cap portion serves as a closure for the open end of the tubular container. Color cosmetic compositions as described herein, such as flowable eyeshadows and blushes, may also be stored and dispensed in either cylindrical containers with a cap/rod/applicator assembly, squeeze tubes, or the like.

In one embodiment, particularly wherein the solvents comprised in the composition are all immiscible with each other or wherein at least one solvent comprised in the composition is immiscible with another solvent comprised in the composition, e.g., wherein the composition has two or more phases, the composition as described herein may be stored and/or packaged within a device comprising a means for emulsifying immiscible solvents, e.g., a ball bearing that mixes the solvents as the device is shaken.

In another embodiment, a cosmetic composition described herein is packaged with instructions for applying the cosmetic composition. In one embodiment, the instructions instruct a consumer to apply a single coat of the cosmetic composition to its intended surface. In another embodiment, the instructions instruct a consumer to apply a single coat of the cosmetic composition to its intended surface with a single stroke. In another embodiment, the instructions instruct the consumer to wait for at least until the composition dries to an even film before application of a subsequent coat.

### EXAMPLES

### Example 1

### Cosmetic Compositions for the Lips

Shown in Table 3 are exemplary single coat lip compositions e.g., lip glosses) comprising at least one high evaporation rate volatile solvent component in an amount of about 5% to about 15% by total weight of the composition, at least one medium evaporation rate volatile solvent component in an amount of about 10% to about 30% by total weight of the composition, and at least one low evaporation rate volatile solvent component in an amount of about 13% to about 30% by total weight of the composition, wherein at least two of the solvents solvate a film former.

TABLE 3

| Description | 1 | 2 | 3 | 4 | 5 |
|---|---|---|---|---|---|
| Butyl Acrylate/ | 5.9 | 4.0 | 5.0 | 3.0 | 5.0 |
| Hydroxypropyldimethicone | | | | | |
| Acrylate Copolymer | | | | | |
| Silicone Acrylate | 13.6 | 18.08 | 19.2 | 16.8 | 17.68 |
| Dimethicone 0.65 cSt | 9 | 16.2 | 14.4 | 12 | 15 |
| (high evaporation volatile | | | | | |
| solvent) | | | | | |
| Dimethicone/trisiloxane blend | 19.0 | 23.0 | 17.0 | 21.0 | 19.7 |
| (medium evaporation volatile | | | | | |
| solvent) | | | | | |
| Isododecane | 11.2 | 4.2 | 7.3 | 7.6 | 3.1 |
| (low evaporation volatile | | | | | |
| solvent) | | | | | |
| Cyclopentasiloxane | 3.7 | 0 | 0.5 | 0 | 0 |
| (low evaporation volatile | | | | | |
| solvent) | | | | | |
| Methyl Trimethicone | 13.1 | 11.4 | 14.3 | 13.7 | 13.4 |
| (low evaporation volatile | | | | | |
| solvent) | | | | | |
| Wetting agent | 1.0 | 1.0 | 1.0 | 1.0 | 1.0 |
| Colorants | 6.5 | 6.5 | 6.5 | 6.5 | 6.5 |
| Fillers | 1.4 | 1.4 | 1.4 | 1.4 | 1.4 |
| Preservative | 1.0 | 1.0 | 1.0 | 1.0 | 1.0 |
| Fragrance | 0.2 | 0.2 | 0.2 | 0.2 | 0.2 |
| Thickener | 5.1 | 0.0 | 1.0 | 0.8 | 0.0 |
| Shine Enhancer | 8.3 | 11.0 | 8.2 | 11.0 | 11.0 |
| Total | 100.0 | 100.0 | 100.0 | 100.0 | 100.0 |

The weight loss of a lip composition as described herein and a prior art lip composition comprising only a low evaporation rate volatile solvent were component compared by measuring the percent weight loss of 0.0355 g of either composition in a thermogravometric analyzer having a platinum pan with a capacity of 60 μl (Perkin Elmer Pyris 1 TGA) at 35° C. over 4 hours (240 minutes). The prior art lip basecoat demonstrated an increased rate of weight loss and more total weight loss over 4 hours, which generally translates to an increased tightening and discomfort on the lips after application (FIG. 1). Additionally, the lip composition as described herein demonstrated rapid weight loss within the first 20 minutes at 35° C. and tower weight loss over time, which generally results in increased comfort on the lips (FIG. 1).

Due to its unique properties, a composition as described herein may exhibit long wear, transfer resistance, and comfort after a single application. Accordingly, unlike prior art long wear, transfer resistant cosmetic compositions, which often require multiple and/or sequential applications of coats (e.g., application of a lipgloss coat followed by application of a topcoat), a long wear, transfer resistant cosmetic composition as described herein, e.g., a lip composition, may be comfortably, and preferably, applied as a single thin coat.

All patents and patent publications referred to herein are hereby incorporated by reference.

Certain modifications and improvements will occur to those skilled in the art upon a reading of the foregoing description. It should be understood that all such modifications and improvements have been deleted herein for the sake of conciseness and readability but are properly within the scope of the following claims.

1-27. (canceled)

28. A cosmetic composition for imparting a long-wearing, comfortable, transfer-resistant film on the lips comprising:
(i) about 1 to about 75% by weight of a high evaporation rate volatile solvent component selected from the group consisting of hexamethyl disiloxane, a silicone fluid having a viscosity of less than 1 cSt at 25° C., and combinations thereof;
(ii) about 1 to about 75% by weight of a medium evaporation rate volatile solvent component;
(iii) about 1 to about 75% by weight of a low evaporation rate volatile solvent component; and
(iv) about 0.1 wt % to about 85 wt % by weight of a silicone acrylate graft copolymer film former that is soluble or dispersible in at least two of the high, medium, and low evaporation rate volatile solvent components;
wherein the cosmetic composition has a flash point of less than about 100° F.

29. The cosmetic composition of claim 28, wherein the silicone acrylate graft copolymer film former is selected from the group consisting of Butyl Acrylate/Hydroxypropyl Dimethicone Acrylate Copolymer (CTFA Monograph ID 12998), Acrylates/Dimethicone Copolymer (CTFA Monograph ID 10082), Acrylates/Ethylhexyl Acrylate/Dimethicone Methacrylate Copolymer (CTFA Monograph ID 16592), and combinations thereof.

30. The cosmetic composition of claim 29, wherein the silicone acrylate graft copolymer film former is selected from the group consisting of Butyl Acrylate/Hydroxypropyl Dimethicone Acrylate Copolymer (CTFA Monograph ID 12998), Acrylates/Dimethicone Copolymer (CTFA Monograph ID 10082), and combinations thereof.

31. The cosmetic composition of claim 30, wherein the film former is present in an amount of about 5 wt % to about 30 wt % by total weight of the composition.

32. The cosmetic composition of claim 28, wherein the high evaporation rate volatile solvent component is present in an amount of about 2.5 wt % to about 50 wt %, the medium evaporation rate volatile solvent component is present in an amount of about 5 wt % to about 50 wt %, and the low evaporation rate volatile solvent component is present in an amount of about 5 wt % to about 50 wt %, by total weight of the composition.

33. The cosmetic composition of claim 32, wherein the high evaporation rate volatile solvent component is selected from the group consisting of hexamethyl disiloxane, dimethicone having a viscosity of about 0.65 cSt, and a combination thereof.

34. The cosmetic composition of claim 33, wherein the high evaporation rate volatile solvent component is dimethicone having a viscosity of 0.65 cSt and is present in an amount of between about 5 wt % to about 15 wt % by total weight of the composition.

35. The cosmetic composition of claim 32 wherein the medium evaporation rate volatile solvent component is a dimethicone/trisiloxane blend.

36. The cosmetic composition of claim 35 wherein the medium evaporation rate volatile solvent component is a dimethicone/trisiloxane blend.

37. The cosmetic composition of claim 32, wherein the low evaporation rate volatile solvent component is selected from the group consisting of cyclopentasiloxane, methyl trimethicone, isododecane, and a combination thereof and is present in an amount of about 13 wt % to about 30 wt % by total weight of the composition.

38. The cosmetic composition of claim 32, wherein the flash point is between about 60° F. and about 100° F.

39. The cosmetic composition of claim 32, wherein the flash point is between about 60° F. and about 65° F.

40. The cosmetic composition of claim 32, wherein the flash point is between about 75° F. and about 80° F.

41. The cosmetic composition of claim 32 wherein the film former is soluble or dispersible in at least the evaporation rate volatile solvent component and the medium evaporation rate volatile solvent component.

42. The cosmetic composition of claim 32, wherein the composition has a viscosity less than 25,000 cps.

43. The cosmetic composition of claim 32, further comprising one or more colorants.

44. A packaged cosmetic comprising a cosmetic composition according to claim 1 and instructions for applying the cosmetic compositions, wherein the instructions instruct a consumer to apply a single coat of the composition to an entire lip with a single stroke.

45. A method for imparting a long-wearing, comfortable, transfer-resistant film on an upper and a lower lip comprising
applying thereto a composition comprising:
(i) about 1 to about 75% by weight of a high evaporation rate volatile solvent component comprising hexamethyl disiloxane, a silicone fluid having a viscosity of less than 1 cSt at 25° C., or a combination thereof;
about 1 to about 75% by weight of a medium evaporation rate volatile solvent component;
(iii) about 1 to about 75% by weight of a low evaporation rate volatile solvent component; and
(iv) about 0.1 wt % to about 85 wt % by weight of a silicone acrylate graft copolymer film former that is soluble or dispersible in at least two of the high, medium, and low evaporation rate volatile solvent components;
wherein the cosmetic composition has a flash point of less than about 100° F., and wherein said applying comprises coating the entire upper lip with said composition using a single stroke and coating the entire lower lip with said composition using a single stroke.
